(19) United States
(12) Patent Application Publication
(10) Pub. No.: US 2019/0091310 A1
(43) Pub. Date: Mar. 28, 2019

(54) NONRELEASED IL-12 FOR THERAPY OF CANCER

(71) Applicants: Stephen E. Wright, Amarillo, TX (US); Hiranmoy Das, Upper Arlington, OH (US)

(72) Inventors: Stephen E. Wright, Amarillo, TX (US); Hiranmoy Das, Upper Arlington, OH (US)

(21) Appl. No.: 16/143,034

(22) Filed: Sep. 26, 2018

Related U.S. Application Data
(60) Provisional application No. 62/563,412, filed on Sep. 26, 2017.

(51) Int. Cl. A61K 39/00; A61K 38/20

(52) U.S. Cl. CPC A61K 39/0011; A61K 38/208

(57) ABSTRACT

The present invention includes compositions and methods comprising: a cancer antigen-specific chimeric antigen receptor cells, e.g., alpha-beta cell receptor T cells, gamma delta cell receptor T cells, induced pluripotent stem cells, hematopoietic stem cells, or natural killer (NK) cells or gamma delta cell receptor T cells, genetically engineered to express non-released IL-12, anchored IL-12, and/or cleavage-resistant IL-12 only, transfected with one or more costimulatory genes; and one or more immune modulators, regulated for safety, in an amount sufficient to eliminate the effect of at least one of myeloid derived suppressor cells (MDSC) or Tregs on the cells or CAR-T cells and eliminate cancer cells.

### CROSS-REFERENCE TO RELATED APPLICATIONS

This application claims priority to U.S. Provisional Application Ser. No. 62/563,412, filed Sep. 26, 2017, the entire contents of which are incorporated herein by reference.

### STATEMENT OF FEDERALLY FUNDED RESEARCH

None.

### TECHNICAL FIELD OF THE INVENTION

The present invention relates in general to the field of therapy for cancer, and more particularly, to a MUC1 CAR-T cell therapy.

### BACKGROUND OF THE INVENTION

Without limiting the scope of the invention, its background is described in connection with therapies for cancer.

One such treatment is taught in U.S. Pat. No. 9,718,887, issued to McColl, et al., entitled “Methods and products for preventing and/or treating metastatic cancer”. Briefly, these inventors are said to teach a method for preventing and/or treating a metastatic cancer in a subject by administering to the subject a therapeutically effective amount of an inhibitor of a chemokine receptor CCX-CKR.

Another such treatment is taught in U.S. Pat. No. 8,658,172, issued to Reinhardt, et al., entitled “Treatment of metastatic breast cancer”, which is said to teach the use of an anti-EpCAM antibody for the manufacture of a medicament for the treatment of metastatic breast cancer.

Yet another such treatment is taught in U.S. Patent Publication No. 20150376296, filed by Fedorov, et al., entitled “Compositions and methods for immunotherapy”. Briefly, these applicants are said to teach the use of immunoresponsive cells, including T cells, cytotoxic T cells, regulatory T cells, and Natural Killer (NK) cells, expressing an antigen recognizing receptor and an inhibitory chimeric antigen receptor (iCAR), and methods of using the immunoresponsive cell for the treatment of neoplasia and other pathologies where an increase in an antigen-specific immune response is desired.

Despite these advances, however, a need remains for improved compositions and methods for the treatment of metastatic cancers.

### SUMMARY OF THE INVENTION

In one embodiment, the present invention includes a composition comprising: a cancer antigen-specific chimeric antigen receptor (CAR) cells or T cell (CAR-T cells) transfected with one or more costimulatory genes; and one or more immune modulators, regulated for safety, in an amount sufficient to eliminate the effect of at least one of myeloid derived suppressor cells (MDSC) or Tregs on the CAR-T cells. In one aspect, the cancer antigen is selected from at least one of: gp100 (MART-1/Melan A), dipeptidyl peptidase IV, adenosine deaminase-binding protein, cyclophilin b, the colorectal cancer antigen C017-1A/GA733, the carcinoembryonic antigen (CEA) and its immunogenic epitopes CAP-1 and CAP-2, etv6, aml1, prostate specific antigen (PSA) and its immunogenic epitopes PSA-1, PSA-2, and PSA-3, prostate-specific membrane antigen (PSMA), T-cell receptor/CD3-zeta chain, the MAGE-family of tumor antigens, the GAGE-family of tumor antigens, BAGE, RAGE, LAGE-1, NAG, GnT-V, MUM-1, CDK4, tyrosinase, p53, MUC family of tumor antigens, HER2/neu, p21ras, RCAS1, α-fetoprotein, E-cadherin, α-catenin, β-catenin, γ-catenin, p120ctn, gp100Pme1117, FRAME, NY-ESO-1, brain glycogen phosphorylase, SSX-1, SSX-2, SSX-1, SSX-4, SSX-5, SCP-1, CT-7, cdc27, adenomatous polyposis coli protein (APC), fodrin, P1A, Connexin 37, Ig-idiotype, p15, gp75, GM2 and GD2 gangliosides, viral products such as human papilloma virus proteins, cyclin dependent kinase-4 (CDK4), BCR-abl, SMAD family of tumor antigens, lmp-1, EBV-encoded nuclear antigen (EBNA)-1, NY-BR-1, NY-BR-62, NY-BR-75, NY-BR-85, NY-BR-87, NY-BR-96, epidermal growth factor receptor (EGFR), Receptor Tyrosine Kinase-Like Orphan Receptor-1 (ROR1), or c-erbB-2. In another aspect, the cells or T cells are selected from at least one of: alpha-beta cell receptor T cells, gamma delta cell receptor T cells, induced pluripotent stem cells, hematopoietic stem cells, or natural killer (NK) cells, gamma delta cell receptor T cells, T cells genetically engineered to express non-released IL-12, anchored IL-12, or cleavage-resistant IL-12 only. In another aspect, the co-stimulatory genes are selected from at least one of CD3 Zeta chain and CD28, 4-1BB, CD28-4-1BB, CD28-OX40, Inducible T-cell CoStimulator (ICOS), or ICOS-4-1BB. In another aspect, the immune modulators that reduce MDSCs are selected from at least one of: IL-12, anchored IL-12, or cleavage-resistant IL-12. In another aspect, the immune modulator that reduces Tregs is Poly-G10.

In one embodiment, the present invention includes a method of adoptive immunotherapy for the treatment of a cancer comprising: obtaining cells from a human subject, wherein the cells are selected from alpha-beta cell receptor T cells, gamma delta cell receptor T cells, induced pluripotent stem cells, hematopoietic stem cells, or natural killer (NK) cells; transfecting the cells with at least one of: a cancer antigen-specific chimeric antigen receptor (CAR) or one or more costimulatory genes to make CAR-T cells; providing one or more immune modulators, regulated for safety, to the subject in an amount sufficient to eliminate at least one of myeloid derived suppressor cells (MDSC) or Tregs; and providing the transfected cells to a subject. In one aspect, the cancer being treated in the subject has an initial diagnosis for recurrence of the cancer. In another aspect, the cancer antigen is selected from at least one of: gp100 (MART-1/Melan A), dipeptidyl peptidase IV, adenosine deaminase-binding protein, cyclophilin b, the colorectal cancer antigen C017-1A/GA733, the carcinoembryonic antigen (CEA) and its immunogenic epitopes CAP-1 and CAP-2, etv6, aml1, prostate specific antigen (PSA) and its immunogenic epitopes PSA-1, PSA-2, and PSA-3, prostate-specific membrane antigen (PSMA), T-cell receptor/CD3-zeta chain, the MAGE-family of tumor antigens, the GAGE-family of tumor antigens, BAGE, RAGE, LAGE-1, NAG, GnT-V, MUM-1, CDK4, tyrosinase, p53, MUC family of tumor antigens, HER2/neu, p21ras, RCAS1, a-fetoprotein, E-cadherin, a-catenin, β-catenin, γ-catenin, p120ctn, gp100Pme1117, FRAME, NY-ESO-1, brain glycogen phosphorylase, SSX-1, SSX-2, SSX-1, SSX-4, SSX-5, SCP-1, CT-7, cdc27, adenomatous polyposis coli protein (APC), fodrin, P1A, Connexin 37, Ig-idiotype, p15, gp75, GM2 and GD2 gangliosides, viral products such as human papilloma virus proteins, cyclin dependent kinase-4 (CDK4), BCR-abl, SMAD family of tumor antigens, lmp-1, EBV-encoded nuclear antigen (EBNA)-1, NY-BR-1, NY-BR-62, NY-BR-75, NY-BR-85, NY-BR-87, NY-BR-96, epidermal growth factor receptor (EGFR), Receptor Tyrosine Kinase-Like Orphan Receptor-1 (ROR1), or c-erbB-2. In another aspect, the co-stimulatory genes are selected from at least one of CD3 Zeta chain and CD28, 4-1BB, CD28-4-1BB, CD28-OX40, Inducible T-cell CoStimulator (ICOS), or ICOS-4-1BB. In another aspect, the immune modulators that reduce MDSCs are selected from at least one of: IL-12, anchored IL-12, or cleavage-resistant IL-12. In another aspect, the immune modulator that reduces Tregs is Poly-G10. In another aspect, the cancer is selected from MUC-1 expressing cancers, such as, colon cancer, breast cancer, ovarian cancer, lung cancer, or pancreatic cancer. In another aspect, the CAR-T cells eliminate the cancer recurrence by at least one of production type I cytokine production, activated T cells, or activation of memory T cells. In another aspect, the CAR and the costimulatory gene are on the same expression vector. In another aspect, the CAR-T cells are activated with antigen presenting dendritic cells, antigen presenting cells, or artificial antigen presenting cells. In another aspect, the CAR-T cells do not trigger systemic inflammatory response syndrome (SIRS), TNF alpha and cytokine release syndrome (CRS), CRP release, IL-6 release, TNF alpha and IL-12, human liver transaminases, Aspartate aminotransferase (AST) release, or Aspartate transaminase (ALT) release. In another aspect, the CAR-T cells trigger a reduced systemic inflammatory response syndrome (SIRS), TNF alpha and cytokine release syndrome (CRS), CRP release, IL-6 release, TNF alpha and IL-12, human liver transaminases, Aspartate aminotransferase (AST) release, or Aspartate transaminase (ALT) release, when compared to CAR-T cells that are not provided with the one or more immune modulators, regulated for safety, to the subject in an amount sufficient to eliminate at least one of myeloid derived suppressor cells (MDSC) or Tregs. In another aspect, the CAR-T cells are activated with artificial antigen presenting cells modified to express IL-15 and IL-21 or IL-7, or exposed to EGTA or Cyclosporin A. In another aspect, the CAR and the one or more costimulatory genes are under the control of an inducible promoter. In another aspect, the CAR-T cells are further treated to prevent expression of TCR-α, β₂-microglobulin, or PD1. In another aspect, the CAR-T cells made resistant to suppressive factors by at least one of: expressing TGF-β in the CAR-T cells a dominant negative (dn) TGF-beta receptor, or inhibiting fas expression. In another aspect, the cells negatively selected for cells that are suppressor/regulatory cells, CD3+/CD4+/Foxp3, induced (i) suppressor/regulatory cells that are CD3+/CD4+ or CD8+ T cells secreting at least one of IL-10 or TGF-beta. In another aspect, the cells are alpha-beta cell receptor T cells, gamma delta cell receptor T cells, induced pluripotent stem cells, hematopoietic stem cells, or natural killer (NK) cells. In another aspect, the cells are gamma delta cell receptor T cells, genetically engineered to express non-released IL-12, anchored IL-12, and/or cleavage-resistant IL-12 only.

### BRIEF DESCRIPTION OF THE DRAWINGS

For a more complete understanding of the features and advantages of the present invention, reference is now made to the detailed description of the invention along with the accompanying figures and in which:

FIG. 1 shows a plasmid for use with the present invention.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

The present inventor has recognized that there is a need for novel therapies for metastatic breast cancer, because almost all patients will die of the disease (1). One such therapy is adoptive T-cell immunotherapy, specifically chimeric antigen receptor (CAR) human T-lymphocytes (T cells) (2), which need to be optimized (3) (4) (5) (6). Aberrant host proteins, such as mucin, expressed by cancer cells may function as tumor antigens. Hypo-glycosylated MUC1 protein is produced by breast cancer cells and is the immune target of interest in this study (7). CAR-T cells directed against such a tumor antigen, Tn, (5E5) eliminated pancreatic cancer in mice, with survival of the mice, however, tumors regrew (8). MAb 5E5 exhibits strict specificity for the secreted Tn MUC1 glycoform, whereas MAb HMFG2 (9) reacts with all glycoforms as well as unglycosylated MUC1 (10-12), thus, HMFG2, may eliminate adenocarcinoma cells not expressing Tn, but may react with normal tissues. An example is a breast cancer cell line, MCF-7, which has a glycosylation pattern that more closely resembles the pattern found in normal epithelial cells (10). MUC1 (HMFG2) CAR-T cells have been shown to eliminate MUC1-expressing human breast cancer cells in immunodeficient mice (9), (13), but did not kill normal mammary gland cells (9), however, the tumors recurred. This may be due to the inadvertent production of T regulatory cells (Treg) during the in vitro development of MUC1 CAR-T cells, which may be eliminated by incubating MUC1 CAR-T cells with Poly-G10 (14, 15). In vivo, this is most likely due to immunosuppression within the tumor microenvironment by myeloid derived suppressor cells (MDSC) (16), which may be overcome by expressing IL-12 by the CAR-T cells (17) (18-20) (21). However, in a mouse melanoma model, inducible (i) IL-18 was less toxic than iIL-12 and enhanced survival (23). With elimination of T regulatory cells and immunosuppression within the immune tumor microenvironment, and T cell inhibitory genes, such as, Fas, PD1, and CTLA-4 (24). With elimination of T regulatory cells and immunosuppression within the immune tumor microenvironment, the optimum costimulatory gene combination (13) may be determined for the elimination of, e.g., breast cancer cells.

The present invention improves adoptive immunotherapy using human MUC1 CAR-T cells. Briefly, the present invention uses immune modulators, regulated for safety, and an optimum costimulatory gene combination, to eliminate MDSC and Treg from the blood and tumor. By eliminating the MDSC and Treg from the blood and tumor there is an increase of T cell elimination of breast cancer cell tumors. The elimination of breast cancer tumors is via type I cytokine production and activated and memory human lymphocytes of non-obese diabetic, severe combined immunodeficient (NOD-scid) common γ-chain gene (γc) negative, beta 2 microglobulin knockout (to prevent graft-versus-host disease (GVHD)) (NSG (NOD-scid-γc (NSG) beta-2-microglobulin (B₂m)) (NSG-B2m)) mice injected with PBMC from the same individual from which the CAR-T cells were generated, a human hepatocyte cell line and human breast cancer cells and human MUC1 CAR-T cells. In another embodiment, the invention includes a knock out TCR alpha constant (TRAC) and knock into TRAC MUC1 CAR-T, knock out TCR beta and knock into TRBC NFAT.hIL-12.γ1.TMCT.PA2, so that expression will only be when CAR-TCR is engaged, and knock out beta 2 microglobulin (required for HLA expression). PD1 will be knocked out to prevent apoptosis. Inducible caspase 9 (icasp9), can be cloned into a transposon plasmid of the sleeping beauty system.

Thus, the present invention includes the generation of MUC1 IL-12 CAR-T cells, from PBMC, by insertion of the gene encoding IL-12 with anchorage and prevention of cleavage, and regulation of expression, to prevent IL-12 systemic toxicity. Moreover, MUC1 IL-12 CAR, coexpressing CD3 Zeta chain and CD28, versus 4-1BB versus CD28-4-1BB versus CD28-OX40, ex vivo, are inserted into human lymphocytes, and compared for safety and efficacy in treatment of mice bearing PBMC from the same individual from which the CAR-T cells were generated, a human hepatocyte cell line (safety) and breast cancer cell tumors (efficacy). In another aspect, CAR, with or without expression of IL-12, coexpressing CD3 Zeta chain and ICOSTM/ICOS/CD28 versus ICOSTM/ICOS/4-1BB (28) versus CD28TM/CD28/4-1BB versus CD28TM/CD28/OX40 (9), ex vivo, is inserted into human lymphocytes, and compared for safety and efficacy in treatment of mice bearing a human hepatocyte cell line (safety) and breast cancer cell tumors (efficacy).

In another aspect, the gene encoding IL-12 will be inserted into the MUC1 CAR-T cells, with a nuclear factor of activated T-cells (NFAT.hIL-12.PA2)-responsive transcription factor that will allow expression only with CAR engagement, insuring expression only when the tumor antigen is engaged. To prevent IL-12 toxicity, IL-12 will be anchored to the cell membrane by B7-1 TM and CT and inhibited from cleavage by inserting IgGHC1 hinge, CH2 and CH3 (γ1) between IL-12 and B7 (NFAT.hIL-12.γ1.TMCT.PA2). The T cells will be converted to universal donor T cells by knocking out TCR alpha constant (TRAC), and knocking into TRAC MUC1 CAR-T, knocking out TCR beta, and knocking into TRBC NFAT.hIL-12.γ1.TMCT.PA2, so that expression will only be when CAR-TCR is engaged, and knocking out beta 2 microglobulin (required for HLA expression) by CRISPR-CAS9 and adeno-associated virus carrying the respective CAR and IL-12 inserts bordered by TRAC or TRBC sequences. PD1 will be knocked out to prevent apoptosis. iCasp9 is cloned into transposon plasmid 26553: pT2/SVNeo and electroporated into the T cells with transposase plasmid p26552: pCMV/SB11.

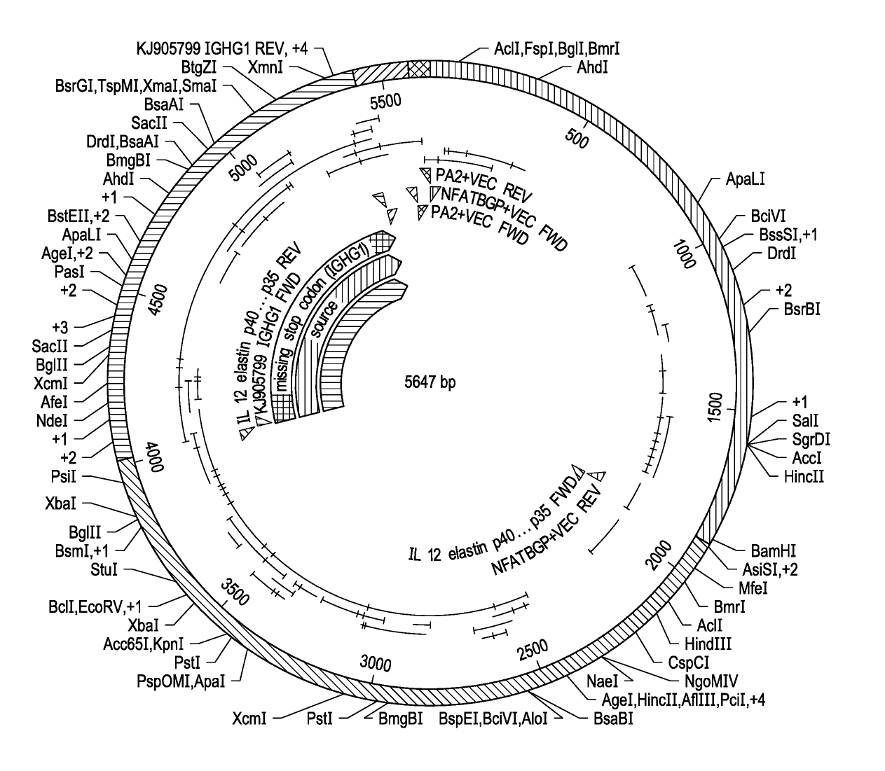

The optimum costimulatory molecule(s) for MUC1 CAR-T lymphocytes will be identified using adoptive immunotherapy. For example, plasmids for the costimulatory combination(s) for the CAR-To produce MUC1 CAR-T cells can be used. In the same cassette, the gene encoding IL-12 with a transcription factor that will allow expression only with CAR engagement, and the genes encoding a CAR and caspase 9, will be cloned into a transposon plasmid of the sleeping beauty system. T cells will be transduced with the sleeping beauty transposon containing IL-12 with a transcription factor, a CAR and caspase 9, along with the sleeping beauty transposase, to generate MUC1 IL-12 CAR-T cells expressing different costimulatory combination(s) (coexpressing CD3 Zeta chain and CD28, versus 4-1BB versus CD28-4-1BB versus CD28-OX40). FIG. 1 shows a plasmid for use with the present invention. In another example, the plasmids for the costimulatory combination(s) for the CAR to produce MUC1 CAR T cells have been obtained. The design knocks out TCR alpha constant (TRAC) and knock into TRAC MUC1 CAR, knock out TCR beta and knock into TRBC internal ribosome entry site (IRES).human interleukin 12 (.hIL-12),IgGHC1 hinge, CH2 and CH3 (.γ1).B7-1 transmembrane (TM) and cytoplasmic tail (CT) (.TMCT).polyA signal sequence (.PA2) (an alternate is nuclear factor of activated T-cells (NFAT).hIL-12.γ1.TMCT.PA2), so that expression will only be when CAR is engaged, and knock out B2m (required for HLA-I expression) and T cell inhibitory genes, such as, Fas, PD1, and CTLA-4. In addition to CAR and IL-12, T cells can be transduced with the sleeping beauty transposon containing inducible caspase 9 (iCasp9), and gRNAs for the above knockouts and the above genes to be knocked in, with bordering sequences to allow homologous recombination with the site of insertion, to generate MUC1 IL-12 CAR T cells expressing different costimulatory combination(s). MUC1 CAR T cells are expanded by the MUC1 gene mutated (to present tumor specific, unglycosylated MUC1) presented by artificial antigen presenting cells (aAPC) expressing IL-21 to maintain undifferentiated lymphocytes and IL-15 to enhance survival and improve proliferation. Safety and efficacy of MUC1 CAR T cells expressing different costimulatory combination(s), with or without expression of IL-12, will be compared in treatment of mice bearing a human hepatocyte cell line, to study hepatocyte toxicity, and breast cancer cell tumors, to study efficacy of tumor cell killing. Human cytokines produced in systemic inflammatory response syndrome (SIRS) (29), e.g., TNF alpha and cytokine release syndrome (CRS) (30), e.g., CRP (IL-6 surrogate (31)), TNF alpha and IL-12, human liver transaminases, AST and ALT, and elimination of breast cancer cell tumors, myeloid derived suppressor cells (MDSC) and T regulatory (Treg) suppressor lymphocytes from the blood and tumor and increase MUC1 CAR T cells killing of breast cancer cells, type I cytokine production and activated and memory lymphocytes of mice injected with a human hepatocyte cell line and human breast cancer cells will be correlated.

In another embodiment, MUC1 CAR-T cells will be expanded by the MUC1 gene mutated (to present tumor specific, unglycosylated MUC1) presented by artificial antigen presenting cells (aAPC) expressing IL-21 to maintain undifferentiated lymphocytes and IL-15 to enhance survival and improve proliferation. Safety and efficacy of MUC1 CAR-T cells expressing different costimulatory combination(s), with or without IL-12, are compared for safety and efficacy in treatment of mice bearing PBMC from the same individual from whom the CAR-T cells were generated, to assay for cytokines produced in cytokine storm, a human hepatocyte cell line, to study hepatocyte toxicity, and breast cancer cell tumors, to study efficacy of tumor cell killing. Human cytokines produced in systemic inflammatory response syndrome (SIRS) (22), e.g., TNF alpha and cytokine release syndrome (CRS) (23), e.g., CRP (IL-6 surrogate (24)), TNF alpha and IL-12, human liver transaminases, AST and ALT, and elimination of breast cancer cell tumors, myeloid derived suppressor cells (MDSC) and (Treg) suppressor lymphocytes from the blood and tumor and increase MUC1 CAR-T cells killing of breast cancer cells, type I cytokine production and activated and memory lymphocytes of mice injected with a human hepatocyte cell line and human breast cancer cells will be correlated.

PBMC from anonymous donors will be the source of T cells. Ex vivo conversion of lymphocytes into MUC1-specific T cells will be performed by introducing a CAR (2) of MUC1 (9). The gene encoding IL-12 (17-19) will be inserted into the MUC1 CAR T cells, with an internal ribosome entry site (IRES) (32-34) (IRES.hIL-12.PA2) (an alternate is a nuclear factor of activated T-cells (NFAT.hIL-12.PA2)-responsive transcription factor that will allow expression only with CAR engagement, insuring expression only when the tumor antigen is engaged (20). To prevent IL-12 toxicity (21), IL-12 will be anchored to the cell membrane by B7-1 TM and CT (35) and inhibited from cleavage by inserting IgGHC1 hinge, CH2 and CH3 (γ1) between IL-12 and B7 (36) (IRES or NFAT.hIL-12.γ1.TMCT.PA2). The T cells will be converted to universal donor T cells by knocking out TCR alpha constant (TRAC) (37), and knocking into TRAC MUC1 CAR (38, 39), knocking out TCR beta (40, 41) and knocking into TRBC IRES or NFAT.hIL-12.γ1.TMCT.PA2, by CRISPR/CAS9 and knocking out B2m and T cell inhibitory genes Fas, PD1, and CTLA-4 (24). Ribonucleotide protein complexes (RNP) composed of gRNAs, under the control of different promoters (24), and Cas9 protein, with genes to be inserted in a transposon (42, 43) (alternatives are single-stranded donor DNA complementary to the nontarget strand (44) or double cut double-stranded donor DNA (45))), will be used since specificity of RNP is superior (46, 47). In addition to MUC1CAR and IL-12, iCasp9 (48, 49) and gRNAs for the above knockouts, under the control of different promoters (24), and the above genes to be knocked in, with bordering sequences to allow homologous recombination with the site of insertion, to generate MUC1 IL-12 CAR T cells expressing different costimulatory combination(s), will be cloned into transposon pT2/SVNeo (42) and electroporated into the T cells with transposase pCMV/SB11 (42, 43). An alternative is adeno-associated virus carrying the respective CAR and IL-12 inserts bordered by TRAC or TRBC sequences (38-41). Assays for MUC1 CAR T and other genes-expressing cells will be as published (43): CAR+ T cells will be detected by staining with human IgG Fcγ. An alternative is a biotinylated MUC1 peptide (24-mer (PAHGVTSAPDTRPAPGSTAPP)) followed by PE-conjugated streptavidin and flow cytometry (9). Expansion of the MUC1 CAR T cells will be performed by using the MUC1 gene mutated to present tumor specific, unglycosylated MUC1 (50), (51), (52), presented by artificial antigen presenting cells (aAPC) (53), expressing IL-15 (54) and IL-21 (55). Reversal of NFAT1 activation will be by inhibition of calcineurin by chelation of extracellular calcium by 2 mM EGTA or, if unsuccessful, addition of 1 microM CsA (56). An alternative is to target MUC1 CAR T cells to the tumor by inserting CCR2 (57). Inactivation of alpha beta (14) and gamma delta (15) Treg cells will be performed by incubating MUC1 CAR T cells with Poly-G10. Another addition, if cure is not achieved, is expression of a dominant negative (dn) TGF-beta receptor, which will allow the MUC1 CAR T cells to become resistant to the antiproliferative effects of TGF-beta and retain their effector function in vivo (58). MUC1 CAR T cells will be cultured in 1B2H, or if not available, X-VIVO 15 (59) medium at 2 million cells per ml, and incubated with irradiated aAPC (53), at ten aAPC to one PBMC, weekly, until a sufficient number of cells is obtained or four weeks maximum for IV infusion. 10̂7 cells and supernatant will be frozen on days 0, twice weekly sampling and at infusion for use in cytotoxicity, immunophenotyping, chemokine and chemokine receptors and cytokine assays. MUC1-specific MUC1 CAR T cells cytotoxicity will be tested by XTT assay (60) to confirm their lack of toxicity toward a human hepatocyte cell line, (FL 62891 (ATCC® CRL-11005™)), in association with hepatic transaminases, AST and ALT, and ability to kill the target MUC1-expressing (61), triple negative breast cancer (62) cell line, BT-20 (ATCC® HTB-19™). Non-MUC1-expressing tumor targets will act as controls for tumor antigen specificity. In parallel studies, antigen-specific cytokine releasing activity among corresponding MUC1 CAR T cells and their subpopulations (i.e. CD45RO (memory); CD4/CD25/FOXP3 or CD152 (regulatory); HLA-DR (activated) will be identified and assessed using intracellular cytokine staining and multiparameter flow cytometry (63). We expect an increase in type 1 cytokine production and MUC1-specific cytotoxicity and a decrease in type 2 cytokines. Supernatant of PBMC and PBMC in culture will be assayed for type 1 stimulatory cytokines (IFN-gamma and TNF-alpha (antigen specific), TGF-beta (regulatory), and the type 2 inhibitory cytokine (IL-10 (suppresses cellular immunity)) and chemokines 2-7 & 9 and their receptors. Intracellular cytokine staining assays will be performed using multiparameter immunofluorescence to detect T cells, on a per cell basis, producing specific cytokines and chemokines. A sample of blood (10 ml) collected at the time of blood collection and at infusion and at sacrifice (1 ml), will be assayed for immunophenotyping using multiparameter flow cytometry and cell differentiation (CD) antigen expression markers. Immunophenotyping will be accomplished by flow cytometry (63) to include CD33+ and CD11b+ myeloid derived suppressor cells (MDSC) (16), T cell subpopulation markers for CD3⁺, CD4⁺, CD8⁺, CD56⁺ and/or NKG2D⁺ (natural killer (NK)), CD56 and CD3 (NKT) cells and/or CD45RO memory and CD45RA naïve T-cells co-expressing CD4 or CD8, and CD4/CD8 double positive T cell subpopulations. The expression levels of the chemokine receptors CCR7 and CCR5 and their association with central memory T cell subpopulations can also be determined. The markers for activated T-lymphocytes will include HLA-DR⁺, CD25^{pos/neg}, and CD69⁺. For regulatory T cell subpopulations (T_{regs}), we will assess natural (n) suppressor/regulatory T-lymphocytes, CD3⁺/CD4⁺/Foxp3 (64). Markers for induced (i) suppressor/regulatory T-lymphocytes will be either CD3⁺/CD4⁺ or CD8⁺ T cells secreting IL-10 (65) and/or TGF-beta (65). Additional markers of suppressor T-lymphocytes, CTLA-4 and GITR will also be analyzed. IFN-gamma production after MUC1 peptide stimulation (1 ug/ml X1) will also be assayed by ELISA, intracellular cytokine staining and RT-PCR to study specificity. These assays have been shown to be more sensitive than using HLA bound antigen (66). Since memory T-cells are the cell type correlating with increased cancer cell killing in mice (67) and humans (68), we anticipate an increase in activated memory T-cells with MUC1 CAR infusion. Alternatively, we would also anticipate a down-regulation in regulatory T cell subpopulations. Immunohistochemistry will be performed on tumors to locate the CAR T cells.

MUC1 CAR T cells, 0.5×10̂6 (20), will be administered IV into up to 12 NSG (69) mice, injected in the mammary gland, or IV, with 5×10̂6 luciferase-expressing BT-20 (ATCC® HTB-19™) cells (70), (71), 10̂7 of a human hepatocyte cell line, (FL 62891 (ATCC® CRL-11005™)) IP, in each group, after tumors become palpable at 5 mm or greater. To prevent SIRS, mice will be given Omega-3 Fatty Acids, 3.3 mg equivalents orally per day beginning one month before CAR T cells (29). Prevention of the CRS and liver toxicity from IL-12 will be by giving cyclosporine A (CsA) at 4 mg/kg/d bid orally, for twice elevated hepatocyte transaminases, AST or ALT, or IL-12 serum level. CsA will be stopped when the transaminases and IL-12 levels normalize (30). If CsA at 4 mg/kg/d bid oral fails to normalize IL-12 serum levels, anti-IL-12/23 p40 monoclonal antibody, ustekinumab, will be administered at 15 microg, subcutaneously (SC) (72). If the above fails to normalize IL-12 serum levels, 12.5 μg, SC, of the BB homodimerizer AP20187, a caspase inducible drug (CID) (Clontech Laboratories) will be administered (73). If there is not >80% cure of mice, the optimum MUC1 CAR T cells with or without 50 microg CpG (PF03512676 CpGDNA adjuvant, known also as CpG 7909 and CpG 2006) (74), subcutaneous, and immune modulation (cholecalciferol at 0.6 IU, sildenafil at 0.7 microg, alendronate at 0.3 microg, and celecoxib at 130 microg oral daily, added to drinking water, based on human dosages with interspecies scaling (75)) can be tested.

Analyses. Data Analysis (76), (77). For proportion analysis, it is expected that 50% of mice will develop detectable tumors (71), and that treatment will result in disappearance of tumors of 80% in the experimental group. Mantel Haenszel chi squared analysis will be used to detect differences in incidence between experimental and control groups. If any cell contains fewer than 5 mice, then Fisher's exact test will be used. Analysis of immunologic responses, % MDSC, and activated, suppressor and memory lymphocytes, as well as, their cytokines, chemokines and chemokine receptors at day 0 and day of MUC1 CAR T cells infusion and at sacrifice, is a secondary objective and will be analyzed using summary statistics due to the researchers past experience with the lack of normality of this data. 12 mice will be entered per group with two groups every two months.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Burstein H J, Harris J R, Morrow M. Malignant tumors of the Breast. In: DeVita V T, Lawrence T S, Rosenberg S A, editors. Cancer Principles & Practices of Oncology. 9th ed. Philadelphia, Pa.: Lippincott-Raven publishers; 2011. p. 1401-46.
2. Dotti G, Gottschalk S, Savoldo B, Brenner M K. Design and development of therapies using chimeric antigen receptor-expressing T cells. Immunological Reviews. 2014; 257(1):107-26. doi: 10.1111/imr.12131.
3. Urba W J, Longo D L. Redirecting T Cells. New England Journal of Medicine. 2011; 365(8):754-7. doi: doi:10.1056/NEJMe1106965.
4. Brentjens R J. CARs and cancers: questions and answers. Blood. 2012; 119(17):3872-3. doi: 10.1182/blood-2012-02-410373.
5. Abken H. Costimulation Engages the Gear in Driving CARs. Immunity. 2016; 44(2):214-6. doi: http://dx.doi.org/10.1016/j.immuni.2016.02.001.
6. Kawalekar Omkar U, O'Connor Roddy S, Fraietta Joseph A, Guo L, McGettigan Shannon E, Posey Jr Avery D, Patel Prachi R, Guedan S, Scholler J, Keith B, Snyder N W, Blair I A, Milone Michael C, June Carl H. Distinct Signaling of Coreceptors Regulates Specific Metabolism Pathways and Impacts Memory Development in CAR T Cells. Immunity. 2016; 44(2):380-90. doi: http://dx.doi.org/10.1016/j.immuni.2016.01.021.
7. Beatson R E, Taylor-Papadimitriou J, Burchell J M. MUC1 immunotherapy. Immunotherapy. 2010; 2(3):305-27. Epub 2010/07/20. doi: 10.2217/imt.10.17. PubMed PMID: 20635898.
8. Posey Jr Avery D, Schwab Robert D, Boesteanu Alina C, Steentoft C, Mandel U, Engels B, Stone Jennifer D, Madsen Thomas D, Schreiber K, Haines Kathleen M, Cogdill Alexandria P, Chen Taylor J, Song D, Scholler J, Kranz David M, Feldman Michael D, Young R, Keith B, Schreiber H, Clausen H, Johnson Laura A, June Carl H. Engineered CAR T Cells Targeting the Cancer-Associated Tn-Glycoform of the Membrane Mucin MUC1 Control Adenocarcinoma. Immunity. 2016; 44(6): 1444-54. doi: http://dx.doi.org/10.1016/j.immuni.2016.05.014.
9. Wilkie S, Picco G, Foster J, Davies D M, Julien S, Cooper L, Arif S, Mather S J, Taylor-Papadimitriou J, Burchell J M, Maher J. Retargeting of Human T Cells to Tumor-Associated MUC1: The Evolution of a Chimeric Antigen Receptor. The Journal of Immunology. 2008; 180(7):4901-9. doi: 10.4049/jimmunol.180.7.4901.
10. Sorensen A L, Reis C A, Tarp M A, Mandel U, Ramachandran K, Sankaranarayanan V, Schwientek T, Graham R, Taylor-Papadimitriou J, Hollingsworth M A, Burchell J, Clausen H. Chemoenzymatically synthesized multimeric Tn/STn MUC1 glycopeptides elicit cancer-specific anti-MUC1 antibody responses and override tolerance. Glycobiology. 2006; 16(2):96-107. doi: 10.1093/glycob/cwj044.
11. Maher J, Wilkie S, Davies David M, Arif S, Picco G, Julien S, Foster J, Burchell J, Taylor-Papadimitriou J. Targeting of Tumor-Associated Glycoforms of MUC1 with CAR T Cells. Immunity. 2016; 45(5):945-6. doi: http://dx.doi.org/10.1016/j.immuni.2016.10.014.
12. Blidner Ada G, Mariño Karina V, Rabinovich Gabriel A. Driving CARs into Sweet Roads: Targeting Glycosylated Antigens in Cancer. Immunity. 2016; 44(6):1248-50. doi: http://dx.doi.org/10.1016/j.immuni.2016.06.010.
13. Anurathapan U, Chan R C, Hindi H F, Mucharla R, Bajgain P, Hayes B C, Fisher W E, Heslop H E, Rooney C M, Brenner M K, Leen A M, Vera J F. Kinetics of Tumor Destruction by Chimeric Antigen Receptor-modified T Cells. Mol Ther. 2014; 22(3):623-33. doi: 10.1038/mt.2013.262.
14. Peng G, Guo Z, Kiniwa Y, Voo K S, Peng W, Fu T, Wang D Y, Li Y, Wang H Y, Wang R F. Toll-like receptor 8-mediated reversal of CD4+ regulatory T cell function. Science. 2005; 309(5739): 1380-4.
15. Peng G, Wang H Y, Peng W, Kiniwa Y, Seo K H, Wang R F. Tumor-infiltrating gammadelta T cells suppress T and dendritic cell function via mechanisms controlled by a unique toll-like receptor signaling pathway. Immunity. 2007; 27(2):334-48. Epub 2007/07/28. doi: 51074-7613(07)00332-9 [pii]10.1016/j.immuni.2007.05.020. PubMed PMID: 17656116.
16. Gabrilovich D I, Ostrand-Rosenberg S, Bronte V. Coordinated regulation of myeloid cells by tumours. Nat Rev Immunol. 2012; 12(4):253-68. Epub 2012/03/23. doi: 10.1038/nri3175. PubMed PMID: 22437938; PMCID: Pmc3587148.
17. Wagner H-J, Bollard C M, Vigouroux S, Huls M R, Anderson R, Prentice H G, Brenner M K, Heslop H E, Rooney C M. A strategy for treatment of Epstein-Barr virus-positive Hodgkin's disease by targeting interleukin 12 to the tumor environment using tumor antigen-specific T cells. Cancer Gene Therapy. 2004; 11(2):81-91. doi: 10.1038/sj.cgt.7700664. PubMed PMID: 12046711.
18. Kerkar S P, Muranski P, Kaiser A, Boni A, Sanchez-Perez L, Yu Z, Palmer D C, Reger R N, Borman Z A, Zhang L, Morgan R A, Gattinoni L, Rosenberg S A, Trinchieri G, Restifo N P. Tumor-specific CD8+ T cells expressing interleukin-12 eradicate established cancers in lymphodepleted hosts. Cancer Res. 2010; 70(17):6725-34. Epub 2010/07/22. doi: 0008-5472.CAN-10-0735 [pii]10.1158/0008-5472.CAN-10-0735. PubMed PMID: 20647327; PMCID: 2935308.
19. Kerkar S P, Goldszmid R S, Muranski P, Chinnasamy D, Yu Z, Reger R N, Leonardi A J, Morgan R A, Wang E, Marincola F M, Trinchieri G, Rosenberg S A, Restifo N P. IL-12 triggers a programmatic change in dysfunctional myeloid-derived cells within mouse tumors. The Journal of Clinical Investigation. 2011; 121(12):4746-57. doi: 10.1172/JCI58814.
20. Zhang L, Kerkar S P, Yu Z, Zheng Z, Yang S, Restifo N P, Rosenberg S A, Morgan R A. Improving Adoptive T Cell Therapy by Targeting and Controlling IL-12 Expression to the Tumor Environment. Mol Ther. 2011; 19(4):751-9. doi: http://www.nature.com/mt/journal/v19/n4/suppinfo/mt2010313 s1.html.
21. Zhang L, Morgan R A, Beane J D, Zheng Z, Dudley M E, Kassim S H, Nahvi A V, Ngo L T, Sherry R M, Phan G Q, Hughes M S, Kammula U S, Feldman S A, Toomey M A, Kerkar S P, Restifo N P, Yang J C, Rosenberg S A. Tumor-infiltrating lymphocytes genetically engineered with an inducible gene encoding interleukin-12 for the immunotherapy of metastatic melanoma. Clin Cancer Res. 2015; 21(10):2278-88. Epub 2015/02/20. doi: 10.1158/1078-0432.ccr-14-2085. PubMed PMID: 25695689; PMCID: Pmc4433819.
22. Hu B, Ren J, Luo Y, Keith B, Young R M, Scholler J, Zhao Y, June C H. Augmentation of Antitumor Immunity by Human and Mouse CAR T Cells Secreting IL-18. Cell Reports. 2017; 20(13):3025-33. doi: https://doi.org/10.1016/j.celrep.2017.09.002.
23. Kunert A, Chmielewski M, Wijers R, Berrevoets C, Abken H, Debets R. Intra-tumoral production of IL18, but not IL12, by TCR-engineered T cells is non-toxic and counteracts immune evasion of solid tumors. Oncoimmunology. 2017; 7(1):e1378842. Epub 2018/01/04. doi: 10.1080/2162402x.2017.1378842. PubMed PMID: 29296541; PMCID: PMC5739571.
24. Ren J, Zhang X, Liu X, Fang C, Jiang S, June C H, Zhao Y. A versatile system for rapid multiplex genome-edited CAR T cell generation. Oncotarget. 2017; 8(10):17002-11. Epub 2017/02/16. doi: 10.18632/oncotarget.15218. PubMed PMID: 28199983; PMCID: PMC5370017.
25. Carpenito C, Milone M C, Hassan R, Simonet J C, Lakhal M, Suhoski M M, Varela-Rohena A, Haines K M, Heitjan D F, Albelda S M, Carroll R G, Riley J L, Pastan I, June C H. Control of large, established tumor xenografts with genetically retargeted human T cells containing CD28 and CD137 domains. Proceedings of the National Academy of Sciences. 2009; 106(9):3360-5. doi: 10.1073/pnas.0813101106.
26. Matsushita M, Plotkin J, Riviere I, Sadelain M. Chimeric Antigen Receptors Combining 4-1BB and CD28 Signaling Domains Augment PI(3)kinase/AKT/Bc1-X(L) Activation and CD8(+) T Cell-mediated Tumor Eradication. Molecular Therapy. 2010; 18(2):413-20. doi: 10.1038/mt.2009.210. PubMed PMID: PMC2839303.
27. Zhong X S, Matsushita M, Plotkin J, Riviere I, Sadelain M. Chimeric antigen receptors combining 4-1BB and CD28 signaling domains augment PI3kinase/AKT/Bcl-XL activation and CD8+ T cell-mediated tumor eradication. Mol Ther. 2010; 18(2):413-20. Epub 2009/09/24. doi: 10.1038/mt.2009.210. PubMed PMID: 19773745; PMCID: PMC2839303.
28. Guedan S, Posey A D, Jr., Shaw C, Wing A, Da T, Patel P R, McGettigan S E, Casado-Medrano V, Kawalekar O U, Uribe-Herranz M, Song D, Melenhorst J J, Lacey S F, Scholler J, Keith B, Young R M, June C H. Enhancing CAR T cell persistence through ICOS and 4-1BB costimulation. JCI Insight. 2018; 3(1). doi: 10.1172/jci.insight.96976.
29. Takatsuka H, Takemoto Y, Iwata N, Suehiro A, Hamano T, Okamoto T, Kanamaru A, Kakishita E. Oral eicosapentaenoic acid for complications of bone marrow transplantation. Bone Marrow Transplantation. 2001; 28(8):769. PubMed PMID: 8889403.
30. Xu X-J, Tang Y-M. Cytokine release syndrome in cancer immunotherapy with chimeric antigen receptor engineered T cells. Cancer Letters. 2014; 343(2):172-8. doi: http://dx.doi.org/10.1016/j.canlet.2013.10.004.
31. Davila M L, Riviere I, Wang X, Bartido S, Park J, Curran K, Chung S S, Stefanski J, Borquez-Ojeda O, Olszewska M, Qu J, Wasielewska T, He Q, Fink M, Shinglot H, Youssif M, Satter M, Wang Y, Hosey J, Quintanilla H, Halton E, Bernal Y, Bouhassira D C, Arcila M E, Gonen M, Roboz G J, Maslak P, Douer D, Frattini M G, Giralt S, Sadelain M, Brentj ens R. Efficacy and toxicity management of 19-28z CAR T cell therapy in B cell acute lymphoblastic leukemia. Sci Transl Med. 2014; 6(224):224ra25. Epub 2014/02/21. doi: 10.1126/scitranslmed.3008226. PubMed PMID: 24553386; PMCID: Pmc4684949.
32. Pegram H J, Purdon T J, van Leeuwen D G, Curran K J, Giralt S A, Barker J N, Brentjens R J. IL-12-secreting CD19-targeted cord blood-derived T cells for the immunotherapy of B-cell acute lymphoblastic leukemia. Leukemia. 2015; 29(2):415-22. doi: 10.1038/leu.2014.215.
33. Koneru M, Purdon T J, Spriggs D, Koneru S, Brentjens R J. IL-12 secreting tumor-targeted chimeric antigen receptor T cells eradicate ovarian tumors in vivo. Oncolmmunology. 2015; 4(3):e994446. doi: 10.4161/2162402X.2014.994446.
34. Yeku O O, Purdon T J, Koneru M, Spriggs D, Brentjens R J. Armored CAR T cells enhance antitumor efficacy and overcome the tumor microenvironment. Scientific Reports. 2017; 7:10541. doi: 10.1038/s41598-017-10940-8. PubMed PMID: PMC5585170.
35. Pan W-Y, Lo C-H, Chen C-C, Wu P-Y, Roffler S R, Shyue S-K, Tao M-H. Cancer Immunotherapy Using a Membrane-bound Interleukin-12 With B7-1 Transmembrane and Cytoplasmic Domains. Mol Ther. 2012; 20(5):927-37. doi: http://www.nature.com/mt/journal/v20/n5/suppinfo/mt201210s1.html.
36. Liao K-W, Chou W-C, Lo Y-C, Roffler S R. Design of transgenes for efficient expression of active chimeric proteins on mammalian cells. Biotechnology and Bioengineering. 2001; 73(4):313-23. doi: 10.1002/bit.1064.
37. Qasim W, Zhan H, Samarasinghe S, Adams S, Amrolia P, Stafford S, Butler K, Rivat C, Wright G, Somana K, Ghorashian S, Pinner D, Ahsan G, Gilmour K, Lucchini G, Inglott S, Mifsud W, Chiesa R, Peggs K S, Chan L, Farzeneh F, Thrasher A J, Vora A, Pule M, Veys P. Molecular remission of infant B-ALL after infusion of universal TALEN gene-edited CAR T cells. Science Translational Medicine. 2017; 9(374). doi: 10.1126/scitranslmed.aaj2013.
38. MacLeod D T, Antony J, Martin A J, Moser R J, Hekele A, Wetzel K J, Brown A E, Triggiano M A, Hux J A, Pham C D, Bartsevich V V, Turner C A, Lape J, Kirkland S, Beard C W, Smith J, Hirsch M L, Nicholson M G, Jantz D, McCreedy B. Integration of a CD19 CAR into the TCR Alpha Chain Locus Streamlines Production of Allogeneic Gene-Edited CAR T Cells. Mol Ther. 2017; 25(4):949-61. Epub 2017/02/27. doi: 10.1016/j.ymthe.2017.02.005. PubMed PMID: 28237835; PMCID: PMC5383629.
39. Eyquem J, Mansilla-Soto J, Giavridis T, van der Stegen S J C, Hamieh M, Cunanan K M, Odak A, Gonen M, Sadelain M. Targeting a CAR to the TRAC locus with CRISPR/Cas9 enhances tumour rejection. Nature. 2017; advance online publication. doi: 10.1038/nature21405 http://www.nature.com/nature/journal/vaop/ncurrent/abs/nature21405.html#supplementary-information.
40. Liu X, Zhang Y, Cheng C, Cheng A W, Zhang X, Li N, Xia C, Wei X, Liu X, Wang H. CRISPR-Cas9-mediated multiplex gene editing in CAR-T cells. Cell Res. 2017; 27(1):154-7. doi: 10.1038/cr.2016.142.
41. Knipping F, Osborn M J, Petri K, Tolar J, Glimm H, von Kalle C, Schmidt M, Gabriel R. Genome-wide Specificity of Highly Efficient TALENs and CRISPR/Cas9 for T Cell Receptor Modification. Molecular Therapy—Methods & Clinical Development. 2017; 4:213-24. doi: 10.1016/j.omtm.2017.01.005.
42. Cui Z, Geurts A M, Liu G, Kaufman C D, Hackett P B. Structure-Function Analysis of the Inverted Terminal Repeats of the Sleeping Beauty Transposon. Journal of Molecular Biology. 2002; 318(5):1221-35. doi: 10.1016/s0022-2836(02)00237-1.
43. Huls M H, Figliola M J, Dawson M J, Olivares S, Kebriaei P, Shpall E J, Champlin R E, Singh H, Cooper L J. Clinical application of Sleeping Beauty and artificial antigen presenting cells to genetically modify T cells from peripheral and umbilical cord blood. Journal of visualized experiments: JoVE. 2013(72):e50070. Epub 2013/02/15. doi: 10.3791/50070. PubMed PMID: 23407473; PMCID: PMC3596954.
44. Richardson C D, Ray G J, DeWitt M A, Curie G L, Corn J E. Enhancing homology-directed genome editing by catalytically active and inactive CRISPR-Cas9 using asymmetric donor DNA. Nature Biotechnology. 2016; 34:339. doi: 10.1038/nbt.3481 https://www.nature.com/articles/nbt.3481#supplementary-information.
45. Zhang J P, Li X L, Li G H, Chen W, Arakaki C, Botimer G D, Baylink D, Zhang L, Wen W, Fu Y W, Xu J, Chun N, Yuan W, Cheng T, Zhang X B. Efficient precise knockin with a double cut HDR donor after CRISPR/Cas9-mediated double-stranded DNA cleavage. Genome biology. 2017; 18(1):35. Epub 2017/02/22. doi: 10.1186/s13059-017-1164-8. PubMed PMID: 28219395; PMCID: PMC5319046.
46. DeWitt M A, Corn J E, Carroll D. Genome editing via delivery of Cas9 ribonucleoprotein. Methods. 2017; 121-122:9-15. doi: https://doi. org/10.1016/j.ymeth.2017.04.003.
47. Komor A C, Badran A H, Liu D R. CRISPR-Based Technologies for the Manipulation of Eukaryotic Genomes. Cell. 2017; 168(1):20-36. doi: https://doi.org/10.1016/j.cell.2016.10.044.
48. Tey S K, Dotti G, Rooney C M, Heslop H E, Brenner M K. Inducible caspase 9 suicide gene to improve the safety of allodepleted T cells after haploidentical stem cell transplantation. Biol Blood Marrow Transplant. 2007; 13(8):913-24. Epub 2007/07/21. doi: 10.1016/j.bbmt.2007.04.005. PubMed PMID: 17640595; PMCID: Pmc2040267.
49. Di Stasi A, Tey S-K, Dotti G, Fujita Y, Kennedy-Nasser A, Martinez C, Straathof K, Liu E, Durett A G, Grilley B, Liu H, Cruz C R, Savoldo B, Gee A P, Schindler J, Krance R A, Heslop H E, Spencer D M, Rooney C M, Brenner M K. Inducible Apoptosis as a Safety Switch for Adoptive Cell Therapy. New England Journal of Medicine. 2011; 365(18):1673-83. doi: doi:10.1056/NEJMoa1106152. PubMed PMID: 22047558.
50. Dombrowski K E, Wright S E. Construction of a multiple mucin tandem repeat with a mutation in the tumor-specific epitope by a solid-phase gene assembly protocol. Nucleic Acids Res. 1992; 20(24):6743-4. Epub 1992/12/25. PubMed PMID: 1282708; PMCID: 334601.
51. Quinlin I S, Burnside J S, Dombrowski K E, Phillips C A, Dolby N, Wright S E. Context of MUC1 epitope: immunogenicity. OncolRep. 2007; 17(2):453-6.
52. Wright S E, Quinlin I S, Rewers-Felkins K A, Dombrowski K E, Phillips C A. Retention of immunogenicity produced by mucin 1 peptides with glycosylation site substitutions. Immunopharmacol Immunotoxicol. 2010; 32(4):647-55. Epub 2010/05/18. doi: 10.3109/08923971003671082. PubMed PMID: 20470225.
53. Singh H, Manuri P R, Olivares S, Dara N, Dawson M J, Huls H, Hackett P B, Kohn D B, Shpall E J, Champlin R E, Cooper L J N. Redirecting specificity of T-cell populations for CD19 using the Sleeping Beauty system. Cancer Res. 2008; 68(8):2961-71.
54. Hoyos V, Savoldo B, Quintarelli C, Mahendravada A, Zhang M, Vera J, Heslop H E, Rooney C M, Brenner M K, Dotti G. Engineering CD19-specific T lymphocytes with interleukin-15 and a suicide gene to enhance their anti-lymphoma/leukemia effects and safety. Leukemia. 2010; 24(6):1160-70. doi: http://www.nature.com/leu/journal/v24/n6/suppinfo/leu201075s1.html.
55. Singh H, Figliola M J, Dawson M J, Huls H, Olivares S, Switzer K, Mi T, Maiti S, Kebriaei P, Lee D A, Champlin R E, Cooper L J N. Reprogramming CD19-Specific T Cells with IL-21 Signaling Can Improve Adoptive Immunotherapy of B-Lineage Malignancies. Cancer Research. 2011; 71(10):3516-27. doi: 10.1158/0008-5472.can-10-3843.
56. Loh C, Shaw K T-Y, Carew J, Viola J P B, Luo C, Perrino B A, Rao A. Calcineurin Binds the Transcription Factor NFAT1 and Reversibly Regulates Its Activity. Journal of Biological Chemistry. 1996; 271(18):10884-91. doi: 10.1074/jbc.271.18.10884.
57. Moon E K, Carpenito C, Sun J, Wang L-C S, Kapoor V, Predina J, Powell D J, Riley J L, June C H, Albelda S M. Expression of a Functional CCR2 Receptor Enhances Tumor Localization and Tumor Eradication by Retargeted Human T cells Expressing a Mesothelin-Specific Chimeric Antibody Receptor. Clinical Cancer Research. 2011; 17(14):4719-30. doi: 10.1158/1078-0432. ccr-11-0351.
58. Bollard C M, Rossig C, Calonge M J, Huls M H, Wagner H J, Massague J, Brenner M K, Heslop H E, Rooney C M. Adapting a transforming growth factor beta-related tumor protection strategy to enhance antitumor immunity. Blood. 2002; 99(9):3179-87. Epub 2002/04/20. PubMed PMID: 11964281.
59. Medvec A R, Ecker C, Kong H, Winters E A, Glover J, Varela-Rohena A, Riley J L. Improved Expansion and In Vivo Function of Patient T Cells by a Serum-free Medium. Molecular Therapy—Methods & Clinical Development. 2018; 8(Supplement C):65-74. doi: https://doi.org/10.1016/j.omtm.2017.11.001.
60. Wright S E, Khaznadar R, Wang Z, Quinlin I S, Rewers-Felkins K A, Phillips C A, Patel S. Generation of MUC1-stimulated mononuclear cells using optimized conditions. Scandllmmunol. 2008; January 67(1):24-9.
61. Kurihara T, Brough D E, Kovesdi I, Kufe D W. Selectivity of a replication-competent adenovirus for human breast carcinoma cells expressing the MUC1 antigen. The Journal of Clinical Investigation.106(6):763-71. doi: 10.1172/JCI9180.
62. Lehmann B D, Bauer J A, Chen X, Sanders M E, Chakravarthy A B, Shyr Y, Pietenpol J A. Identification of human triple-negative breast cancer subtypes and preclinical models for selection of targeted therapies. The Journal of Clinical Investigation. 2011; 121(7):2750-67. doi: 10.1172/JCI45014.
63. Dobrzanski M J, Rewers-Felkins K A, Quinlin I S, Samad K A, Phillips C A, Robinson W, Dobrzanski D J, Wright S E. Autologous MUC1-specific Th1 effector cell immunotherapy induces differential levels of systemic TReg cell subpopulations that result in increased ovarian cancer patient survival. Clinlmmunol. 2009; 133:333-52.
64. Jiang H, Chess L. An integrated view of suppressor T cell subsets in immunoregulation. JClinlnvest. 2004; 114(9):1198-208.
65. Vieira P L, Christensen J R, Minaee S, O'Neill E J, Barrat F J, Boonstra A, Barthlott T, Stockinger B, Wraith D C, O'Garra A. IL-10-secreting regulatory T cells do not express Foxp3 but have comparable regulatory function to naturally occurring CD4+CD25+ regulatory T cells. J Immunol. 2004; 172(10):5986-93.
66. Coulie P G, Karanikas V, Colau D, Lurquin C, Landry C, Marchand M, Dorval T, Brichard V, Boon T. A monoclonal cytolytic T-lymphocyte response observed in a melanoma patient vaccinated with a tumor-specific antigenic peptide encoded by gene MAGE-3. ProcNatlAcadSciUSA. 2001; 98(18):10290-5.
67. Dobrzanski M J, Reome J B, Dutton R W. Type 1 and type 2 CD8+ effector T cell subpopulations promote long-term tumor immunity and protection to progressively growing tumor. J Immunol 2000 Jan. 15; 164(2):916-252000.
68. Gold J E, Ross S D, Krellenstein D J, LaRosa F, Malamud S C, Osband M E. Adoptive transfer of ex vivo activated memory T-cells with or without cyclophosphamide for advanced metastatic melanoma: results in 36 patients. EurJCancer. 1995; 31A(5):698-708.
69. Shultz L D, Ishikawa F, Greiner D L. Humanized mice in translational biomedical research. NatRevlmmunol. 2007; 7(2):118-30.
70. Tong A W, Papayoti M H, Netto G, Armstrong D T, Ordonez G, Lawson J M, Stone M J. Growth-inhibitory Effects of CD40 Ligand (CD154) and Its Endogenous Expression in Human Breast Cancer. Clinical Cancer Research. 2001; 7(3):691-703.
71. Wright S E, Rewers-Felkins K A, Quinlin I S, Eldridge P W, Zorsky P E, Klug P P, Phillips C A, Philip R. Adoptive immunotherapy of mucinl expressing adenocarcinomas with mucinl stimulated human peripheral blood mononuclear cells. IntJMolMed. 2002; 9(4):401-4.
72. Ritchlin C, Rahman P, Kavanaugh A, McInnes I B, Puig L, Li S, Wang Y, Shen Y-K, Doyle M K, Mendelsohn A M, Gottlieb A B, Group obotPS. Efficacy and safety of the anti-IL-12/23 p40 monoclonal antibody, ustekinumab, in patients with active psoriatic arthritis despite conventional non-biological and biological anti-tumour necrosis factor therapy: 6-month and 1-year results of the phase 3, multicentre, double-blind, placebo-controlled, randomised PSUMMIT 2 trial. Annals of the Rheumatic Diseases. 2014; 73(6):990-9. doi: 10.1136/annrheumdis-2013-204655.
73. Diaconu I, Ballard B, Zhang M, Chen Y, West J, Dotti G, Savoldo B. Inducible Caspase-9 Selectively Modulates the Toxicities of CD19-Specific Chimeric Antigen Receptor-Modified T Cells. Molecular Therapy. 2017; 25(3):580-92. doi: 10.1016/j.ymthe.2017.01.011. PubMed PMID: PMC5363196.
74. Wright S E, Rewers-Felkins K A, Chowdhury N I, Ahmed J, Srivastava S K. Prevention of human adenocarcinoma with CpG-ODN in a mouse model. Oncology Letters. 2012. Epub In press.
75. Mahmood I. Interspecies scaling of maximum tolerated dose of anticancer drugs: relevance to starting dose for phase I clinical trials. AmJTher. 2001; 8(2):109-16.
76. Davis C S. Statistical Methods for the Analysis of Repeated Measurements. New York: Springer-Verlag; 2002. 1-156 p.
77. Piantadosi S. Clinical Trails, A Methodologic Perspective. New York: John Wiley & Sones, inc; 1997. 1-159 p.
78. Skeen M J, Ziegler H K. Activation of gamma delta T cells for production of IFN-gamma is mediated by bacteria via macrophage-derived cytokines IL-1 and IL-12. The Journal of Immunology. 1995; 154(11):5832-41.
79. Liu Z, Eltoum I-E A, Guo B, Beck B H, Cloud G A, Lopez R D. Protective Immunosurveillance and Therapeutic Antitumor Activity of γδ T Cells Demonstrated in a Mouse Model of Prostate Cancer. The Journal of Immunology. 2008; 180(9):6044-53. doi: 10.4049/jimmunol.180.9.6044.
80. Gomes A Q, Martins D S, Silva-Santos B. Targeting γδ T Lymphocytes for Cancer Immunotherapy: From Novel Mechanistic Insight to Clinical Application. Cancer Research. 2010; 70(24):10024-7. doi: 10.1158/0008-5472.Can-10-3236.
81. Aggarwal R, Lu J, Kanji S, Das M, Joseph M, Lustberg M B, Ray A, Pompili V J, Shapiro C L, Das H. Human Vγ2Vδ2 T cells limit breast cancer growth by modulating cell survival □, apoptosis□related molecules and microenvironment in tumors. International Journal of Cancer. 2013; 133(9):2133-44. doi: doi:10.1002/ijc.28217.
82. Lu J, Das M, Kanji S, Aggarwal R, Joseph M, Ray A, Shapiro C L, Pompili V J, Das H. Induction of ATM/ATR pathway combined with Vγ2Vδ2 T cells enhances cytotoxicity of ovarian cancer cells. Biochimica et Biophysica Acta (BBA)—Molecular Basis of Disease. 2014; 1842(7): 1071-9. doi: https://doi. org/10.1016/j.bbadis.2014.04.003.
83. Legut M, Cole D K, Sewell A K. The promise of γδ T cells and the γδ T cell receptor for cancer immunotherapy. Cellular And Molecular Immunology. 2015; 12:656. doi: 10.1038/cmi.2015.28.
84. Zysk A, DeNichilo M O, Panagopoulos V, Zinonos I, Liapis V, Hay S, Ingman W, Ponomarev V, Atkins G, Findlay D, Zannettino A, Evdokiou A. Adoptive transfer of ex vivo expanded Vγ9Vδ2 T cells in combination with zoledronic acid inhibits cancer growth and limits osteolysis in a murine model of osteolytic breast cancer. Cancer Letters. 2017; 386:141-50. doi: https://doi.org/10.1016/j.canlet.2016.11.013.
85. June C H, O'Connor R S, Kawalekar O U, Ghassemi S, Milone M C. CAR T cell immunotherapy for human cancer. Science. 2018; 359(6382):1361-5. doi: 10.1126/science.aar6711.

What is claimed is:

1. A composition comprising:
a cancer antigen-specific chimeric antigen receptor (CAR) cells or T cell (CAR-T cells) transfected with one or more costimulatory genes; and
one or more immune modulators, regulated for safety, in an amount sufficient to eliminate the effect of at least one of myeloid derived suppressor cells (MDSC) or Tregs on the CAR-T cells.

2. The composition of claim 1, wherein the cancer antigen is selected from at least one of: gp100 (MART-1/Melan A), dipeptidyl peptidase IV, adenosine deaminase-binding protein, cyclophilin b, the colorectal cancer antigen C017-1A/GA733, the carcinoembryonic antigen (CEA) and its immunogenic epitopes CAP-1 and CAP-2, etv6, aml1, prostate specific antigen (PSA) and its immunogenic epitopes PSA-1, PSA-2, and PSA-3, prostate-specific membrane antigen (PSMA), T-cell receptor/CD3-zeta chain, the MAGE-family of tumor antigens, the GAGE-family of tumor antigens, BAGE, RAGE, LAGE-1, NAG, GnT-V, MUM-1, CDK4, tyrosinase, p53, MUC family of tumor antigens, HER2/neu, p21ras, RCAS1, α-fetoprotein, E-cadherin, α-catenin, β-catenin, γ-catenin, p120ctn, gp100Pme1117, FRAME, NY-ESO-1, brain glycogen phosphorylase, SSX-1, SSX-2, SSX-1, SSX-4, SSX-5, SCP-1, CT-7, cdc27, adenomatous polyposis coli protein (APC), fodrin, P1A, Connexin 37, Ig-idiotype, p15, gp75, GM2 and GD2 gangliosides, viral products such as human papilloma virus proteins, cyclin dependent kinase-4 (CDK4), BCR-abl, SMAD family of tumor antigens, lmp-1, EBV-encoded nuclear antigen (EBNA)-1, NY-BR-1, NY-BR-62, NY-BR-75, NY-BR-85, NY-BR-87, NY-BR-96, epidermal growth factor receptor (EGFR), Receptor Tyrosine Kinase-Like Orphan Receptor-1 (ROR1), or c-erbB-2.

3. The composition of claim 1, wherein the cells are selected from at least one of: alpha-beta cell receptor T cells, gamma delta cell receptor T cells, induced pluripotent stem cells, hematopoietic stem cells, or natural killer (NK) cells, gamma delta cell receptor T cells, T cells genetically engineered to express non-released IL-12, anchored IL-12, or cleavage-resistant IL-12 only.

4. The composition of claim 1, wherein the co-stimulatory genes are selected from at least one of CD3 Zeta chain and CD28, 4-1BB, CD28-4-1BB, CD28-OX40, Inducible T-cell CoStimulator (ICOS), or ICOS-4-1BB.

5. The composition of claim 1, wherein the immune modulators that reduce MDSCs are selected from at least one of: IL-12, anchored IL-12, or cleavage-resistant IL-12.

6. The composition of claim 1, wherein the immune modulator that reduces Tregs is Poly-G10.

7. A method of adoptive immunotherapy for the treatment of a cancer comprising:
obtaining cells from a human subject, wherein the cells are selected from alpha-beta cell receptor T cells, gamma delta cell receptor T cells, induced pluripotent stem cells, hematopoietic stem cells, or natural killer (NK) cells;
transfecting the cells with at least one of: a cancer antigen-specific chimeric antigen receptor (CAR) or one or more costimulatory genes to make CAR-T cells;
providing one or more immune modulators, regulated for safety, to the subject in an amount sufficient to eliminate at least one of myeloid derived suppressor cells (MDSC) or Tregs; and
providing the transfected cells to a subject.

8. The method of claim 7, wherein the cancer being treated in the subject has an initial diagnosis for recurrence of the cancer.

9. The method of claim 7, wherein the cancer antigen is selected from at least one of: gp100 (MART-1/Melan A), dipeptidyl peptidase IV, adenosine deaminase-binding protein, cyclophilin b, the colorectal cancer antigen C017-1A/GA733, the carcinoembryonic antigen (CEA) and its immunogenic epitopes CAP-1 and CAP-2, etv6, aml1, prostate specific antigen (PSA) and its immunogenic epitopes PSA-1, PSA-2, and PSA-3, prostate-specific membrane antigen (PSMA), T-cell receptor/CD3-zeta chain, the MAGE-family of tumor antigens, the GAGE-family of tumor antigens, BAGE, RAGE, LAGE-1, NAG, GnT-V, MUM-1, CDK4, tyrosinase, p53, MUC family of tumor antigens, HER2/neu, p21ras, RCAS1, α-fetoprotein, E-cadherin, α-catenin, β-catenin, γ-catenin, p120ctn, gp100Pme1117, FRAME, NY-ESO-1, brain glycogen phosphorylase, SSX-1, SSX-2, SSX-1, SSX-4, SSX-5, SCP-1, CT-7, cdc27, adenomatous polyposis coli protein (APC), fodrin, P1A, Connexin 37, Ig-idiotype, p15, gp75, GM2 and GD2 gangliosides, viral products such as human papilloma virus proteins, cyclin dependent kinase-4 (CDK4), BCR-abl, SMAD family of tumor antigens, lmp-1, EBV-encoded nuclear antigen (EBNA)-1, NY-BR-1, NY-BR-62, NY-BR-75, NY-BR-85, NY-BR-87, NY-BR-96, epidermal growth factor receptor (EGFR), Receptor Tyrosine Kinase-Like Orphan Receptor-1 (ROR1), or c-erbB-2.

10. The method of claim 7, wherein the co-stimulatory genes are selected from at least one of CD3 Zeta chain and CD28, 4-1BB, CD28-4-1BB, CD28-OX40, Inducible T-cell CoStimulator (ICOS), or ICOS-4-1BB.

11. The method of claim 7, wherein the immune modulators that reduce MDSCs are selected from at least one of: IL-12, anchored IL-12, or cleavage-resistant IL-12.

12. The method of claim 7, wherein the immune modulator that reduces Tregs is Poly-G10.

13. The method of claim 7, wherein the cancer is selected from MUC-1 expressing cancers, such as, colon cancer, breast cancer, ovarian cancer, lung cancer, or pancreatic cancer.

14. The method of claim 7, wherein the CAR-T cells eliminate the cancer recurrence by at least one of production type I cytokine production, activated T cells, or activation of memory T cells.

15. The method of claim 7, wherein the CAR and the costimulatory gene are on the same expression vector.

16. The method of claim 7, wherein the CAR-T cells are activated with antigen presenting dendritic cells, antigen presenting cells, or artificial antigen presenting cells.

17. The method of claim 7, wherein the CAR-T cells do not trigger systemic inflammatory response syndrome (SIRS), TNF alpha and cytokine release syndrome (CRS), CRP release, IL-6 release, TNF alpha and IL-12, human liver transaminases, Aspartate aminotransferase (AST) release, or Aspartate transaminase (ALT) release.

18. The method of claim 7, wherein the CAR-T cells trigger a reduced systemic inflammatory response syndrome (SIRS), TNF alpha and cytokine release syndrome (CRS), CRP release, IL-6 release, TNF alpha and IL-12, human liver transaminases, Aspartate aminotransferase (AST) release, or Aspartate transaminase (ALT) release, when compared to CAR-T cells that are not provided with the one or more immune modulators, regulated for safety, to the subject in an amount sufficient to eliminate at least one of myeloid derived suppressor cells (MDSC) or Tregs.

19. The method of claim 7, wherein the CAR-T cells are activated with artificial antigen presenting cells modified to express IL-15 and IL-21 or IL-7, or exposed to EGTA or Cyclosporin A.

20. The method of claim 7, wherein the CAR and the one or more costimulatory genes are under the control of an inducible promoter.

21. The method of claim 7, wherein the CAR-T cells are further treated to prevent expression of TCR-α, β₂-microglobulin, or PD1.

22. The method of claim 7, wherein the CAR-T cells made resistant to suppressive factors by at least one of: expressing TGF-β in the CAR-T cells a dominant negative (dn) TGF-beta receptor, or inhibiting fas expression.

23. The method of claim 7, wherein the cells negatively selected for cells that are suppressor/regulatory cells, CD3+/CD4+/Foxp3, induced (i) suppressor/regulatory cells that are CD3+/CD4+ or CD8+ T cells secreting at least one of IL-10 or TGF-beta.
